(19) United States
(12) Patent Application Publication
(10) Pub. No.: US 2024/0262836 A1
(43) Pub. Date: Aug. 8, 2024

(54) NOVEL MTOR INHIBITOR COMPOUNDS

(71) Applicant: Galderma Holding S.A., Zug (CH)

(72) Inventors: Craig Steven HARRIS, Biot (FR); Emmanuel VIAL, La Colle sur Loup (FR); Ugo ZANELLI, Pisa (I) (IT)

(73) Assignee: Galderma Holding S.A., Zug (CH)

(21) Appl. No.: 18/590,624

(22) Filed: Feb. 28, 2024

Related U.S. Application Data
(63) Continuation of application No. PCT/IB22/57943, filed on Aug. 24, 2022.
(60) Provisional application No. 63/239,353, filed on Aug. 31, 2021.

(51) Int. Cl. C07D 487/04; A61K 31/519

(52) U.S. Cl. CPC C07D 487/04; A61K 31/519

(57) ABSTRACT

Novel compounds having formula (I) and methods of using these compounds to treat diseases, conditions, and disorders are described.

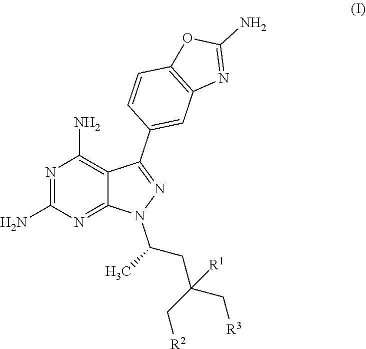

### CROSS-REFERENCE TO RELATED APPLICATION

This application is a continuation of International Patent Application No. PCT/IB2022/057943, filed Aug. 24, 2022, which application claims priority under 35 U.S.C. § 119(e) to U.S. Provisional Application No. 63/239,353, filed Aug. 31, 2021, the entire contents of all of which are incorporated herein by reference.

### TECHNICAL FIELD

The present technology generally relates to novel mTOR inhibitor compounds. Particularly, the present technology relates to identification, preparation and use of a process for synthesis of novel of mTOR inhibitor compounds.

### BACKGROUND

The mammalian target of rapamycin (mTOR) is a multidomain protein and a member of the family of phosphoinositide (PI) 3-kinase-related kinases [PIKKs]. The protein kinase mTOR is the catalytic center of two functionally distinct multiprotein complexes, conserved in all eukaryotes and named mTORC1 and mTORC2. mTOR is notably known for regulating cell proliferation, cell growth, cell mobility, cell survival, protein biosynthesis and transcription. mTOR inhibitors such as Rapamycin are useful pharmaceutical compounds for causing disruptions of the mTOR signaling pathway. They have a central role in the regulation of cell growth and are useful for preventing, controlling and treating a variety of diseases and pathological conditions, including dermatological conditions and cancer treatment.

Novel mTOR inhibitor compounds are disclosed in PCT publication WO2019/122059, for example, and have the following general formula (I):

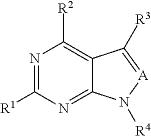

PCT publication WO2019/122065 is also known, which more particularly describes mTOR-inhibiting bicyclic compounds, also used in dermatological complaints and cancer treatment. The entire teaching of these documents are incorporated herein by reference in their entirety.

The mTOR pathway has been found to play a fundamental role in regulating many major cellular processes and is implicated in an increasing number of pathological conditions. Altogether, mTOR activation leads to increased synthesis of multiple proteins, and there is increasing evidence suggesting that its deregulation is associated with human diseases, including cancer and diabetes. Therefore, agents that selectively modulate mTOR production and activity are of great interest as therapeutic targets for the treatment of various diseases.

### SUMMARY

The present disclosure provides novel compounds having formula (I)

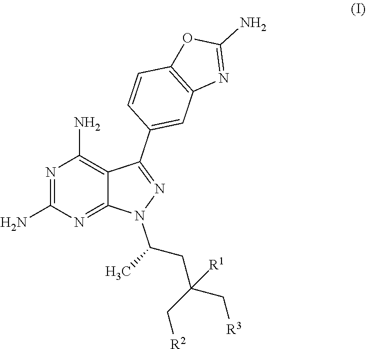

wherein R¹, R² and R³ are each independently selected from hydrogen or OH.

The present disclosure also provides salts and enantiomers, including pharmaceutically acceptable salts and enantiomers, of the compound of formula (I). The present disclosure also provides compositions and pharmaceutical compositions comprising the compound of formula (I) and a carrier or a pharmaceutically acceptable carrier.

The compounds disclosed herein are mTOR inhibitors. Accordingly, in one aspect, provided are pharmaceutical compositions comprising the compound of formula (I), a salt thereof, or an enantiomer thereof and methods of using the compound of formula (I), a salt thereof, or an enantiomer thereof for the treatment of diseases, disorders, or conditions associated with mTOR activity. The compounds are intended to be used as a medicament, in particular in the treatment of diseases involving an mTOR enzyme with serine-threonine kinase activity and notably in the treatment of dermatological complaints associated with a keratinization disorder with a proliferative, inflammatory and/or immunoallergic component, such as psoriasis, atopic dermatitis, actinic keratosis or acne, preferably atopic dermatitis, more preferably the inflammatory component of atopic dermatitis and even more preferably topical treatment of the inflammatory component of atopic dermatitis. Thus, in another aspect, the present disclosure provides methods of treating a disease, disorder, or condition involving mTOR with serine-threonine kinase activity in a subject, wherein the method comprises administering to the subject, a pharmaceutical composition comprising the compound of formula (I).

### BRIEF DESCRIPTION OF THE DRAWINGS

FIGS. 1A-1D show LC-UV chromatograms at 280 nm of CD14547 M1, CD14547 M2, CD14547 M3 and PolyCYP359 sample, respectively.

FIGS. 2A-2D depict graphs showing the mTOR inhibitory activity of CD14547, CD14547 M1, CD14547 M2 and CD14547 M3, respectively.

FIGS. 3A-3C depict graphs showing the mTOR inhibitory activity of pool A, pool B and pool C, respectively.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

Unless the context indicates otherwise, it is specifically intended that the various features of the invention described herein can be used in any combination. Moreover, the disclosure also contemplates that in one or more embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

### Definitions

As used herein, “about” will be understood by persons of ordinary skill in the art and will vary to some extent depending upon the context in which it is used. If there are uses of the term which are not clear to persons of ordinary skill in the art, given the context in which it is used, such as before a numerical designation, e.g., temperature, time, amount, and concentration, including range, indicates approximations which may vary by (+) or (−) 10%, 5% or 1%.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein may be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The expression “comprising” means “including, but not limited to.” For example, compositions and methods include the recited elements, but do not exclude others. “Consisting essentially of” shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “mTOR inhibitor” refers to compounds which down-regulate, i.e. reduce, block or even suppress, the activation of the mTOR signaling pathway, by competing, advantageously selectively, with the substrates at the level of mTORC1 and/or mTORC2 or by modifying the active site of these enzymes which can thus no longer catalyze a given substrate. The terms (mTOR) “antagonist” and (mTOR) “inhibitor” are used without preference according to the present invention.

As used herein, the term “derivatives” means both the metabolic derivatives thereof and the chemical derivatives thereof.

In some embodiments, isomers of the compounds disclosed herein are contemplated. These include, both stereoisomers and constitutional or structural isomers. As used herein, the term “stereoisomer” refers to both enantiomers and diastereomers. Constitutional or structural isomers include, for example, regioisomers, which are compounds having the same functional group, but attached at different positions.

As used herein, “treating” or “treatment” of a disease in a patient refers to (1) preventing the symptoms or disease from occurring in an animal that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, the term treatment excludes prevention or prophylaxis.

As used herein, the term “subject” is used interchangeably with “patient,” and indicates a mammal, or a human, ovine, bovine, feline, canine, equine, simian, etc. Non-human animals subject to diagnosis or treatment include, for example, simians, murine, such as, rat, mice, canine, leporid, livestock, sport animals, and pets. In one or more embodiments, the subject is a human.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks. Consistent with this definition and as used herein, the term “therapeutically effective amount” is an amount sufficient to treat a specified disorder or disease or alternatively to obtain a pharmacological response.

“Pharmaceutically acceptable” means in the present description being useful in preparing a pharmaceutical composition that is generally safe, non-toxic and neither biologically nor otherwise undesirable and includes being useful for veterinary use as well as human pharmaceutical use.

“Pharmaceutically acceptable salts” or “salts thereof” mean salts which are pharmaceutically acceptable, as defined above, and which possess the desired pharmacological activity. Such salts include acid addition salts formed with organic and inorganic acids, such as hydrogen chloride, hydrogen bromide, hydrogen iodide, sulfuric acid, phosphoric acid, acetic acid, glycolic acid, maleic acid, malonic acid, oxalic acid, methanesulfonic acid, trifluoroacetic acid, fumaric acid, succinic acid, tartaric acid, citric acid, benzoic acid, ascorbic acid and the like. Base addition salts may be formed with organic and inorganic bases, such as sodium, ammonia, potassium, calcium, ethanolamine, diethanolamine, N-methylglucamine, choline and the like. Included are pharmaceutically acceptable salts or compounds of any of the Formulae herein.

Depending on its structure, the phrase “pharmaceutically acceptable salt,” as used herein, refers to a pharmaceutically acceptable organic or inorganic acid or base salt of a compound. Representative pharmaceutically acceptable salts include, e.g., alkali metal salts, alkali earth salts, ammonium salts, water-soluble and water-insoluble salts, such as the acetate, amsonate (4,4-diaminostilbene-2, 2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The present disclosure provides novel compounds which are useful as modulators of the mTOR protein kinase. These novel compounds are therefore potential active ingredients for a variety of therapeutic the treatment of pathological conditions which involves modulation of mTOR production and/or activity.

### Compounds

The mammalian TOR (mTOR) pathway is a key regulator of cell growth and proliferation. The mTOR pathway integrates signals from nutrients, energy status and growth factors to regulate many processes, including autophagy, ribosome biogenesis and metabolism. Recent work identifying upstream regulators of mTOR has revealed that mTOR can sense diverse signals and produce a myriad of responses. Despite these developments, there is still a need for more effective agents for the treatment of diseases. The inventors have now discovered a series of compounds that are potent mTOR inhibitors and are thus useful in therapy of mTOR mediated disorders.

Accordingly an object of the present disclosure is to provide novel mTOR inhibitor compounds for use in the treatment of a variety of potential indications. The compounds, compositions and methods of the present disclosure are advantageous compared to other topical mTOR inhibitors in that they have higher efficacy, improved safety, and can be formulated for innovative dosing schedules and delivery methods.

The inventors discovered that the compounds described herein are metabolites of the compound (S)-3-(2-aminobenzo[d]oxazol-5-yl)-1-(4-methylpentan-2-yl)-1H-pyrazolo[3,4-d]pyrimidine-4,6-diamine (CD14547) which has the following structure:

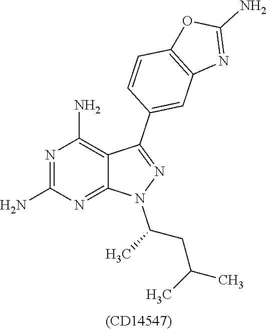 [SMILES: CC(C)C[C@H](C)n1nc(-c2ccc3oc(N)nc3c2)c2c(N)nc(N)nc21]

Particularly, the present disclosure relates to novel mTOR compounds, to the process for synthesizing such compounds, and to the use of the compounds in pharmaceutical compositions for the treatment of diseases, conditions, and disorders. The compounds of the present disclosure act as modulators, e.g., inhibitors of protein kinase mTOR. They are consequently of use in the treatment of mTOR mediated diseases, conditions, or disorders.

Novel compounds of formula (I) provided herein exhibit a good mTOR-inhibiting activity. Thus, the present disclosure provides compounds of formula (I) below:

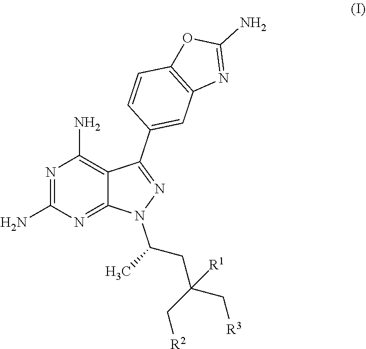

a pharmaceutically acceptable salt thereof, or an enantiomer thereof;
wherein R¹, R² and R³ are each independently selected from H or OH.

In some embodiments, the present disclosure provides compounds of formula (I) wherein: when R¹ is OH, R² and R³ are each independently H, when R² is OH, R¹ and R³ are each independently H, and when R³ is OH, R¹ and R² are each independently H.

In other embodiments, the present disclosure provides compounds of formula (I) wherein:

| Compound | Formula | R¹ | R² | R³ |
|---|---|---|---|---|
| M1 | II | OH | H | H |
| M2 | III | H | OH | H |
| M3 | IV | H | H | OH |

In another aspect, the present disclosure provides compounds of formula (II) below:

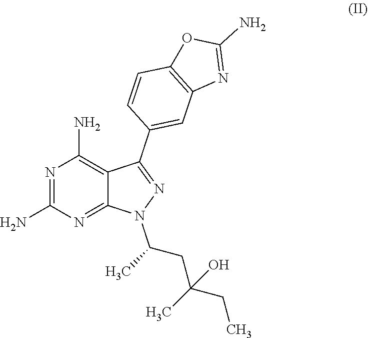 [SMILES: CCC(C)(O)C[C@H](C)n1nc(-c2ccc3oc(N)nc3c2)c2c(N)nc(N)nc21]

a salt thereof, or an enantiomer thereof.

In another aspect, the present disclosure provides compounds of formula (III) below:

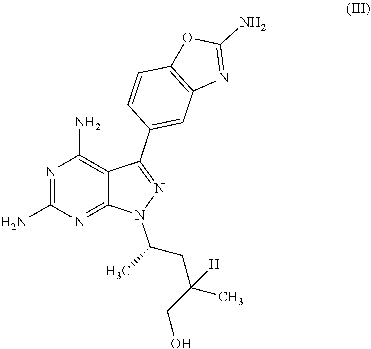 [SMILES: CC(CO)C[C@H](C)n1nc(-c2ccc3oc(N)nc3c2)c2c(N)nc(N)nc21]

a salt thereof, or an enantiomer thereof.

In another aspect, the present disclosure provides compounds of formula (IV) below:

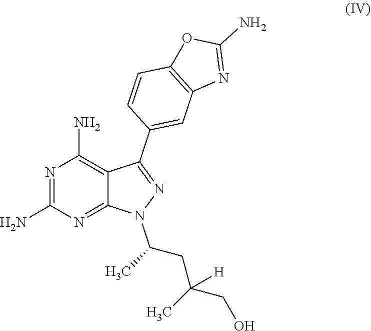 [SMILES: CC(CO)C[C@H](C)n1nc(-c2ccc3oc(N)nc3c2)c2c(N)nc(N)nc21]

a salt thereof, or an enantiomer thereof.

In some embodiments, the present disclosure provides salts of the compound of formula (I), formula (II) formula (III) or formula (IV). Moreover, the salts are pharmaceutically and/or physiologically acceptable salts of the compound of formula (I), formula (II) formula (III) or formula (IV). Moreover, the present disclosure provides enantiomers, in particular pharmaceutically acceptable enantiomers, of the compound of formula (I), formula (II) formula (III) or formula (IV).

The present disclosure not only provides the compound of formula (I), formula (II) formula (III) or formula (IV) per se, but also its pharmaceutically acceptable salts, solvates, hydrates, esters, amides, stereoisomers, derivatives, polymorphs and prodrugs thereof, and also its various crystalline and amorphous forms. The pharmaceutically acceptable salts may include salts of the compounds of formula (I), formula (II) formula (III) or formula (IV) formed with a pharmaceutically acceptable acid and salts of the compounds of formula (I), formula (II) formula (III) or formula (IV) formed with a pharmaceutically acceptable base, such as identified above.

### Compositions

Provided herein are pharmaceutical compositions comprising, consisting of, or consisting essentially of compound of formula (I), formula (II) formula (III) or formula (IV), or an equivalent thereof. In any embodiments, the present disclosure provides a pharmaceutical composition comprising one or more of the compound of formula (I), formula (II) formula (III) or formula (IV) or a pharmaceutically acceptable salt thereof as described herein, and a pharmaceutically acceptable carrier and/or excipient. In any embodiments, the pharmaceutical composition comprises a therapeutically effective amount of the compound of formula (I), formula (II) formula (III) or formula (IV), or a pharmaceutically acceptable salt thereof, and a pharmaceutically acceptable carrier or excipient. Suitable pharmaceutical carriers and excipients include those which are pharmaceutically acceptable and compatible with the selected method of administration. The pharmaceutical compositions described herein may contain various carriers or excipients known to those skilled in the art.

Suitable pharmaceutically acceptable excipients and carriers are generally known to those skilled in the art and are thus included in the present disclosure. Pharmaceutical compositions of compound of formula (I) or an equivalent thereof of the present disclosure can be prepared as formulations according to standard methods and using excipients and carriers. A pharmaceutically acceptable carrier includes such carriers as, for example, aqueous solutions, non-toxic excipients including salts, preservatives, buffers and the like, which are described in Remington's Pharmaceutical Sciences, 15th Ed. Easton: Mack Publishing Co., pp. 1405-1412 and 1461-1487 (1975), The National Formulary XIV., 14th Ed. Washington: American Pharmaceutical Association (1975) and Remington's Pharmaceutical Science, Mark Publishing Co., New Jersey (1991), all of which are incorporated herein by reference. Exemplary carriers or excipients may include, but are not limited to, emollients, ointment base, emulsifying agents, solubilizing agents, humectants, thickening or gelling agents, wetting agents, texture enhancers, stabilizers, pH regulators, osmotic pressure modifiers, emulsifiers, UV-A and UV-B screening agents, preservatives, permeation enhancer, chelating agents, antioxidants, acidifying agents, alkalizing agents, buffering agents and vehicle or solvent. The compounds prepared by the methods described herein can be formulated prior to administration. The selection of the formulation should be decided by the attending physician taking into consideration the same factors involved with determining the effective amount.

The compounds prepared by the methods described herein can be converted in to its pharmaceutically acceptable salts using the methods known in the art. The salts can be produced before or after the isolation of the particular compound. Thus in one embodiment, pharmaceutically acceptable salts of compound of Formula I, II, III or IV can be prepared.

The present technology also relates to pharmaceutically acceptable derivatives, including salts and pro-drugs, tautomers, solvates, and hydrates of the compounds described herein. The compounds of Formula I, II, III or IV may exist as solvates, especially hydrates. Hydrates may form during manufacture of the compounds or compositions comprising the compounds, or hydrates may form over time due to their hygroscopic nature. Compounds of Formula I, II, III or IV may exist as organic solvates as well, including DMF, ether, and alcohol solvates among others. The identification and preparation of any particular solvate is within the skill of the ordinary artisan of synthetic organic or medicinal chemistry.

An aspect of the present invention is also a composition comprising, in a pharmaceutically acceptable medium, a compound of Formula I, II, III or IV as defined above or a pharmaceutically acceptable salt thereof. A pharmaceutically acceptable medium denotes a medium that is compatible with and suitable for use in contact with human and animal cells, in particular with the skin, mucous membranes and/or the integuments, without undue toxicity, irritation or allergic response or the like, and commensurate with a reasonable benefit/risk ratio. A pharmaceutically acceptable medium according to the invention may comprise any known adjuvant used in the pharmaceutical field, which is compatible with the mTOR-inhibiting compounds according to the present technology. Suitable examples of pharmaceutically acceptable medium include, but are not limited to solvents, buffers, aromatizing agents, binders, chelating agents, surfactants, thickeners, lubricants, gellants, humectants, moisturizers, preserving agents, antioxidants, calmative agents, pro-penetrating agents, colorants, fragrances and the like, or a mixture thereof. Other optional components, known to a person skilled in the art can be added to the pharmaceutical composition such that the advantageous properties intrinsically associated with the present invention are not, or are not substantially, adversely affected by the envisaged addition at a concentration so that they not harm the advantageous properties of the compounds.

The pharmaceutical compositions of the present technology may be in liquid, solid or gas form and may be administered orally, rectally, buccally, intranasaly, topically, transdermally, by intra-arterial injection, intraperitoneally, or parenterally (subcutaneously, intramuscularly or intravenously), intravaginally, as an inhalant or via an impregnated or coated device such as a stent. Other administration methods are also contemplated. In one or more embodiments, the pharmaceutical compositions are administered topically. For administration via oral route, the composition may be in the form of tablets, gel capsules, coated tablets, syrups, suspensions, solutions, powders, granules, emulsions, suspensions of microspheres or nanospheres or lipid or polymeric vesicles allowing controlled release. For administration via parenteral route, the composition may be in the form of solutions or suspensions for perfusion or for injection. For administration via the topical route, the compositions, which are thus more particularly intended for treating the skin and mucous membranes, may be in liquid, pasty or solid form, and more particularly in the form of ointments, creams, milks, pomades, powders, impregnated pads, syndets, solutions, gels, sprays, mousses, lotions, suspensions, sticks, shampoos or washing bases. They may also be in the form of suspensions of microspheres or nanospheres or lipid or polymeric vesicles, or of polymeric or gelled patches, or of hydrogels allowing controlled release of the active compounds. These topical compositions may moreover be either in anhydrous form or in an aqueous form.

The pharmaceutical composition according to the technology may include between 0.001% and 10%, including about 0.001% to about 5%, about 0.001% to about 3%, about 0.001% to about 2%, or about 0.001% to about 1%, of said compound of Formula I, II, III or IV or a pharmaceutically acceptable salt thereof, by weight relative to the total weight of the composition. The amount effectively administered to be used according to the invention depends on the desired therapeutic effect, and may thus vary within a wide range. A person skilled in the art, in particular a medical practitioner, can readily, on the basis of his general knowledge, determine the appropriate amounts. The composition may further include one or more of other active ingredients, such as antibiotics, antibacterials, antivirals, antiparasitic agents, antifungal agents, anesthetics, analgesics, antiallergic agents, retinoids, free-radical scavengers, antipruriginous agents, antihistamines, immunosuppressants, corticosteroids, keratolytic agents, intravenous immunoglobulins, antiangiogenic agents, antiinflammatory agents, and the like, or a combination thereof.

The present disclosure provides compositions, in particular pharmaceutical compositions, comprising one or more compounds of formula (I), formula (II) formula (III) or formula (IV) for the treatment of mTOR mediated diseases, condition, or disorders. The present disclosure provides for the use of at least one compound of formula (I), formula (II) formula (III) or formula (IV) for preparing a pharmaceutical composition in which the compound has mTOR-inhibiting activity.

### Methods of Treatment

In another aspect, provided are pharmaceutical compositions according to the present technology for use as a medicament, in particular in the treatment of diseases involving an mTOR enzyme with serine-threonine kinase activity in a patient. In one or more embodiments, the pharmaceutical compositions may be administered after one or more symptoms have developed. In one or more embodiments, the pharmaceutical compositions may be administered as a preventive measure, for preventing or stopping the progression of a disease or a disorder. In other embodiments, the pharmaceutical compositions may be administered in the absence of symptoms. For example, the pharmaceutical compositions may be administered to a predisposed individual before the appearance of the symptoms (for example in the light of a history of symptoms and/or of genetic factors or other predisposing factors). The treatment may also be continued after the disappearance of the symptoms, for example to prevent or delay their reappearance.

The compound of Formula I prepared by the methods described herein may be used, in a pharmaceutical composition for treating a number of conditions by administering to a subject, such as a human being in need thereof. For example, the compound of Formula I may be used for treating a conditions, for which mTOR inhibitors are known to be effective, such as dermatological complaints and cancer.

In one aspect, the pharmaceutical compositions according to the present technology are particularly intended to be used in the treatment of dermatological complaints associated with a keratinization disorder with a proliferative, inflammatory and/or immunoallergic component. The dermatological complaints associated with a keratinization disorder with a proliferative, inflammatory and/or immunoallergic component comprise keratinization conditions or disorders relating to cell proliferation, notably common acne, comedones, polymorphs, acne rosacea, nodulocystic acne, acne conglobata, senile acne, and secondary acnes such as solar acne, medication-related acne or occupational acne, other keratinization disorders, notably ichthyosis, ichthyosiform conditions, Darier's disease, palmoplantar keratoderma, leukoplakia and leukoplakiform conditions, and cutaneous or mucous (buccal) lichen, other dermatological complaints associated with a keratinization disorder with an inflammatory and/or immunoallergic component, notably all forms of psoriasis, whether it is cutaneous, mucous or ungual psoriasis, and even psoriatic rheumatism, or cutaneous atopy, such as atopic dermatitis (or atopic eczema) or respiratory atopy or gingival hypertrophy, all dermal or epidermal proliferations, whether benign or malignant, and whether of viral origin or otherwise, such as common warts, flat warts and verruciform epidermodysplasia, oral or florid papillomatoses, and lesions or proliferations that may be induced by ultraviolet radiation, notably in the case of actinic keratoses, and basal cell and spinal cell epithelioma. In one or more embodiments, the pharmaceutical compositions are intended to be used in the treatment of dermatological complaints associated with a keratinization disorder with a proliferative, inflammatory and/or immunoallergic component, such as psoriasis, atopic dermatitis, actinic keratosis or acne, even more preferentially atopic dermatitis. In one or more embodiments, the pharmaceutical compositions are intended to be used in the treatment of the inflammatory component of atopic dermatitis, and preferentially the topical treatment of the inflammatory component of atopic dermatitis. The term “inflammatory component of atopic dermatitis” means an inflammation involving the CD4+ lymphocytes, eosinophils, mastocytes and Th2 cytokines. Suitable conditions, for which mTOR inhibitor is known to be effective, include but not limited to including cancer treatment, such as breast cancer, lung cancer, non-small-cell lung cancer, kidney cancer, renal carcinoma, prostate cancer, blood cancer, liver cancer, ovarian cancer, thyroid cancer, endometrial cancer, lymphoma, renal cell carcinoma, or mantle cell lymphoma.

The present technology relates to novel mTOR-inhibiting compounds of Formula I, II, III or IV. Thus, one aspect of the present invention is the compounds of Formula I, II, III or IV as described above which are intended to be used as medicaments. An aspect of the invention is also a composition according to the invention for its use as a medicament, in particular in the treatment of diseases involving an mTOR enzyme with serine-threonine kinase activity in a patient.

An effective amount of compound of Formula I, II, III or IV can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. The dosage can be determined by a physician and adjusted, as necessary, to suit observed effects of the treatment.

In one or more embodiments, a compound of Formula I, II, III or IV is administered thrice daily, twice daily, once daily, every other day, twice per week, three times per week, four times per week, five times per week, six times per week, once per week, once every two weeks, once every three weeks, once every four weeks, once every five weeks, once every six weeks, once every seven weeks, once every eight weeks, once every nine weeks, once every 10 weeks, once every 11 weeks, once every 12 weeks, twice per year, once per year, or any range including and/or in-between any two of these values, and/or as needed.

The treatments have a variable duration, depending on the patient and the therapy. The treatment period may thus run from several days to several years. In one or more embodiments, the duration of treatment is about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about one week, about 2 weeks, about 3 weeks, about 4 weeks, about 5 weeks, about 10 weeks, about 20 weeks, about 30 weeks, about 36 weeks, about 40 weeks, about 48 weeks, about 50 weeks, about one year, about two years, about three years, about four years, about five years, or any range including and/or in-between any two of these values, and/or as needed.

### Methods of Preparation

The present technology also encompasses the preparation of pharmaceutically acceptable salts, solvates, hydrates, esters, amides, stereoisomers, derivatives, polymorphs, prodrugs, and crystalline or amorphous forms of the compounds disclosed herein. Any or all of the compounds set forth in any of the reaction schemes herein may be converted to a pharmaceutically acceptable salt by reaction with an inorganic or organic acid or inorganic or organic base under appropriate conditions known to one skilled in the art. Pharmaceutically acceptable esters and amides can be prepared by reacting, respectively, a hydroxy or amino functional group with a pharmaceutically acceptable organic acid, such as identified above. A prodrug is a drug which has been chemically modified and may be biologically inactive at its site of action, but which is degraded or modified by one or more enzymatic or other in vivo processes to the parent bioactive form. Generally, a prodrug has a different pharmacokinetic profile than the parent drug such that, for example, it is more easily absorbed, it has better salt formation or solubility and/or it has better systemic stability.

The m-Tor inhibitor compounds described herein can be prepared by methods well known in the art of organic chemistry. The starting material used for the synthesis of these compounds can be either synthesized or obtained from commercial sources such but not limited Sigma-Aldrich Company. The compounds described and other related compounds having different substituents are optionally synthesized using techniques and such as for example, in Fieser & Fieser's Reagents for Organic Synthesis, Volumes 1-17 (John Wiley and Sons, 1991); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein. The synthetic procedures described herein, especially when taken with the general knowledge in the art, provide sufficient guidance to those of ordinary skill in the art to perform the synthesis, isolation, and purification of the compounds of the present invention. Further, it is contemplated that the individual features of these embodiments and examples may be combined with the features of one or more other embodiments or examples.

It will also be appreciated by those skilled in the art that in the processes described below the functional groups of intermediate compounds may need to be protected by suitable protecting groups. Protecting groups may be added or removed in accordance with standard techniques, which are known to one of ordinary skill in the art and as described herein. The use of protecting groups is described in detail in Green, T. W. and P. G. M. Wuts, Protective Groups in Organic Synthesis (1999), 3rd Ed., Wiley.

In addition, at any point in any of the reaction schemes disclosed herein, the starting material, an intermediate or a product so formed may be subjected to a resolution process whereby individual enantiomers or diastereomers are separated into starting materials, intermediates or products that are in stereoisomerically substantially pure form. These individual enantiomers, diastereomers or mixtures thereof, can then be used in the method disclosed in any of the reaction schemes herein to prepare stereoisomerically substantially pure forms of the compounds of Formula I, II, III or IV, or mixtures thereof. Methods for resolution of racemates or other stereoisomeric mixtures are well known in the art (e.g., E. L. Eliel and S. H. Wilen, in Stereochemistry of Organic Compounds; John Wiley & Sons: New York, 1994; Chapter 7, and references cited therein).

Methods for preparation of CD14547 are described in U.S. 63/220,820, which is incorporated herein by reference. The following examples illustrate illustrative methods for illustrative compounds provided herein. These examples are not intended, nor are they to be construed, as limiting the scope of the disclosure. It will be clear that the methods can be practiced otherwise than as particularly described herein and for other compounds within the scope of the genus described herein. Numerous modifications and variations are possible in view of the teachings herein and, therefore, are within the scope of the disclosure.

### EXAMPLES

Various embodiments will be further clarified by the following examples, which are in no way intended to limit this disclosure thereto.

### General Abbreviations

° C. Degree Celsius
cm Centimeter
COSY Correlated Spectroscopy
Da Dalton
DMSO Dimethyl Sulfoxide
EGF Epidermal Growth Factor
ELSD Evaporative Light Scattering Detector
g Gram
G6P Glucose-6-Phosphate
G6PDH Glucose-6-Phosphate Dehydrogenase
μg Microgram
h Hour
H₂O Water
HMBC Heteronuclear Multiple Bond Correlation
HP-β-CD 2-Hydroxypropyl-β-Cyclodextrin
HRMS High Resolution Mass Spectrometry
HSQC Heteronuclear Single Quantum Coherence
HTRF Head Related Transfer Function
Hz Hertz
IC50 Half Maximal Inhibitory Concentration
KPi Potassium Phosphate
KPsi Kilo Pounds per Square Inch
L Liter
LC Liquid Chromatography
μL Microliter
m/z Mass-to-charge Ratio
MeCN Acetonitrile
MeOH Methanol
mg Milligram
MgCl₂ Magnesium Chloride
MHz Mega Hertz
min Minute
mL Milliliter
mM Millimolar
mm Millimeter
μm Micrometer
n/a Not applicable
NADP+ Nicotinamide Adenine Dinucleotide Phosphate
nd Not detected
nm Nanometer
nM Nanomolar
NMR Nuclear magnetic Resonance
PDA PhotoDiode Array
ppm Parts-per-million
QA Quality Assurance
rpm Rotation per minute
s Second
see Second
TK Toxicokinetic
UPLC Ultra Performance Liquid Chromatography
UV UltraViolet
V Volume

### General Methods for the Preparation and Characterization of the Compounds

Representative compounds of Formula (I) were synthesized and characterized as described below.

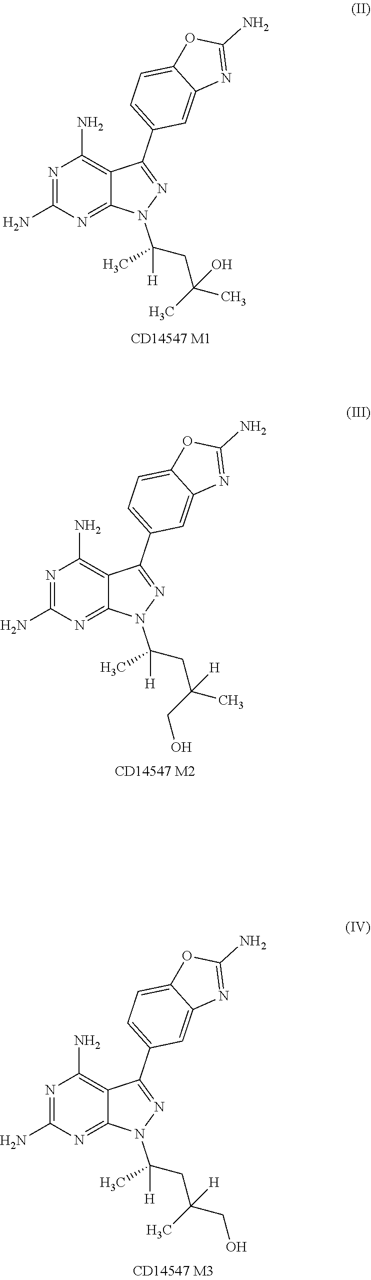 [SMILES: C[C@@H](CC(C)(C)O)n1nc(-c2ccc3oc(N)nc3c2)c2c(N)nc(N)nc21 ; CC(CO)C[C@H](C)n1nc(-c2ccc3oc(N)nc3c2)c2c(N)nc(N)nc21 ; CC(CO)C[C@H](C)n1nc(-c2ccc3oc(N)nc3c2)c2c(N)nc(N)nc21]

### Example 1: Enzymatic Preparation

The reactions were performed in a V-well 96-well polypropylene microtiter plate at 100 μL reaction volume. The reaction comprised 10 μL cofactor reagent stock solution (1 nM G6P, 0.2 mM NADP+, 0.2 UN/mL G6PDH, 0.1 mM MgCl₂ and 100 mM potassium phosphate buffer at pH 7.5 dissolved in cold H₂O), 89.6 μL Poly CYP359 enzyme (from 500 μL stock prepared in cold H₂O to give a final buffer concentration of 100 mM potassium phosphate & 5 mM MgCl₂), 0.4 μL CD14547 (from 12.5, 25, 50 or 75 mg/mL stock in DMSO) to give final concentrations of 50, 100, 200 or 300 mg/L respectively. The highest concentration 300 mg/L was also pre-formulated in the reaction well with 20% 2-hydroxypropyl-β-cyclodextrin (HP-β-CD) prior to addition of the remaining reagents. Reactions were shaken at 200 rpm on a Kuhner (AG Switzerland) 5 cm orbital shaker at 27° C. for 18 hours and stopped by addition of an equal volume of MeCN.

The dose escalation experiments for PolyCYP359 (results reported in Table 1 below) showed that increasing the dose of CD14547 resulted in a concomitant increase in the production of the desired compounds.

TABLE 1
Results for the dose escalation experiment of CYP359
dosed with CD14547 (values are mg/250 mL, quantified
from a standard curve of CD14547).

| | CD14547 | CD14547 M1 | CD14547 M2 | CD14547 M3 |
|---|---|---|---|---|
| Dose | (10.55 min) | (5.79 min) | (6.09 min) | (6.22 min) |
| (mg/L) | 368 m/z | 383 m/z | 383 m/z | 383 m/z |
| 300 | 23.0 | 29.7 | 7.7 | 3.8 |
| 200 | 6.7 | 23.7 | 5.9 | 2.7 |
| 100 | 0.7 | 14.9 | 3.7 | 1.8 |
| 50 | 0.4 | 4.2 | 1.2 | 0.5 |

The total reaction volume (250 mL) was prepared using fresh enzyme materials generated from frozen pellet materials as follows; previously prepared fed-batch-derived cell pellets of a recombinant E. coli strain expressing PolyCYP359 were thawed and re-suspended with phosphate buffer (100 mM KPi at pH8 plus 5 mM MgCl₂) to give a total volume of 350 mL to which 5.6 μL benzonase was added. The cellular suspensions were homogenized using a cell disruptor (1.1 Kw system, Constant Systems Ltd, UK) set at 20 KPsi, then again at 24 KPsi and finally 30 KPsi. The lysates were centrifuged at 47500×g and the crude extract (supernatant) used for the reactions. Fresh stock solutions of CD14547 at 75 mg/mL were prepared by dissolving in DMSO. The reaction comprised 230 mL crude extract, 1.1 mL CD14547 stock solution, 13.6 mL 20% HP-β-CD, and 27.2 mL co-factor solution (50 mM G6P, 10 mM NADP+ and 10 UN/mL G6PDH, all dissolved in 100 mM KPi buffer at pH8) to provide a total volume of271.9 mL. The reactions were transferred to sufficient 250 mL Erlenmeyer flasks as ca. 50 mL aliquots and incubated overnight at 27° C. and 180 rpm (5 cm diameter orbit). The reactions were checked by LC-MS and harvested after 18 hours by stopping the reaction with addition of an equal volume of MeCN, and stored at −80° C. until ready for onward processing.

### Example 3: Extraction and Purification

The CYP359 reaction mixture was thawed at room temperature and centrifuged followed by removal of the upper MeCN layer by siphoning. The lower aqueous phase was re-extracted with an equal amount of MeCN, frozen at −20° C. then the MeCN decanted and combined with the first MeCN phase. This was repeated twice more and the combined MeCN phases concentrated under vacuum and lyophilized.

The dried extract was chromatographed over a Waters XSelect CSH C18 column (5 μm, 19 mmi.d×100 mm, plus a guard column 19 mm i.d.×10 mm), using a MeCN/H2O gradient (both containing +0.1% formic acid) starting at 5/95% holding for 1 minute, then increasing to 18/82 over 7 minutes then further to 34/66 over 5 minutes followed by wash and re-equilibration steps, all at a flow rate of 17 mL/minute. Typical elution times were: pool A, 1.8 to 6.6 min; CD14547 M1, 6.6 7.4 min; CD14547 M2+M3, 7.4 9.0 min; pool B, 9.0 to 11.4 min and pool C, 11.4 to 12.2 min. All pools were lyophilized separately with pool B yielding 591.3 mg and pool C yielding 21.5 mg. The initial yield of pool A was ca. 6 g; however, this was due to extensive amounts of HP-β-CD which was minimized as described below.

The dried pool A material was redissolved in 15 mL MeOH, centrifuged and the supernatant chromatographed over a column of Sephadex LH-20 (4.0 cm i.d.×40 cm) pre-swollen in and eluted with MeOH. The first of the unidentified compounds eluted at 1.3×bed volume at which point collection started and continued until 2.8× bed volume. Concentration of this fraction under vacuum followed by lyophilization yielded 1.56 g pool A.

The CD 1457 M1 (compound of Formula II) fraction was chromatographed on a Waters Atlantis T3 column (5 μm, 19 mm i.d.×100 mm, plus a guard column 19 mm i.d.×10 mm) and eluting with a MeCN/H2O (both containing +0.1% formic acid) gradient starting at 5/95, held for 1 minute, then increased to 18/82 over 7 minutes, then further to 20/80 over 5 minutes followed by wash and re-equilibration steps, all at a flow rate of 17 mL/minute. M1 eluted as a broad peak between 8.2 and 9.8 min, but still contained a small quantity of HP-β-CD that had been used to solubilize CD14547 at 300 mg/L. This was removed from the reaction on a small column of Sephadex LH-20 (2 cm i.d.×17 cm) preswollen and eluted with MeOH. The HP-β-CD eluted between 0.8 and 1.1 bed volumes and M1 between 1.3 and 2.1 bed volumes. Drying and lyophilization of the M1 fraction yielded 24.7 mg CD14547 M1 at 95.6% purity by LC-UV-ELSD.

The CD14547 M2 and M3 containing fraction from the primary fractionation step was initially passed through a column of Sephadex LH-20 (2 cm i.d.×17 cm) pre-swollen in and eluted with MeOH. M2 and M3 eluted away from the residual HP-β-CD between 1.3 and 2.7 bed volumes. This fraction was concentrated under vacuum and chromatographed on a Waters SunFire C18 column (5 μm, 10 mm i.d×150 mm) at 35° C., using a MeCN/H₂O (both containing +0.1% formic acid) gradient starting at 10/90, increased to 15/85 over 1 minute, then again to 19/81 over 10 minutes followed by wash and re-equilibration steps all at a flow rate of 4 mL/minute. M2 eluted between 8.4 and 8.8 minutes and M3 eluted between 9.0 and 9.4 minutes. Direct lyophilization of these fractions yielded 3.5 mg CD14547 M2 at 99.4% purity by LC-UV-ELSD and 2.1 mg CD14547 M3 at 92.8% purity by LC-UV-ELSD.

Aliquots of M1, 2, and 3 (ca. 1 mg each) were used for acquisition of appropriate NMR data. Rough calculations indicated pool A contained ca 3.2 mg related compounds described herein, pool B contained 1.2 mg and pool C, 5.9 mg.

### Example 4: LC-UV-HRMS Method

The LC-UV-HRMS analysis was conducted using a Waters Acquity UPLC H-class set up using the following parameters:

| Pre-column | Phenomenex SecurityGuard Ultra | | |
|---|---|---|---|
| | cartridges for EVO C18 | | |
| Column | Phenomenex Kinetex EVO C18 | | |
| | 2.6 μm; 100 × 2.1 mm | | |
| Solvent A | Water + 0.1% formic acid | | |
| Solvent B | Methanol + 0.1% formic acid | | |
| Wash solvent | Acetonitrile/Methanol/Isopropanol/Water | | |
| | (1 V/1 V/1 V/1 V) + 0.1% formic acid | | |
| Purge solvent | Water/Methanol (9 V/1 V) | | |
| Seal wash | Every 7.5 min with Water/Methanol | | |
| | (9 V/1 V) | | |
| Flow (mL · min⁻¹) | 0.4 | | |
| Gradient | Time (min) | % A | % B |
| | 0.0 | 90 | 10 |
| | 0.5 | 90 | 10 |
| | 9.0 | 75 | 25 |
| | 11.5 | 10 | 90 |
| | 13.5 | 10 | 90 |
| | 13.6 | 90 | 10 |
| | 15.0 | 90 | 10 |

| | |
|---|---|
| Run time (min) | 15.0 |
| Column temperature (° C.) | 45 |
| Sample manager | 15 |
| temperature (° C.) | |

The samples were analyzed using a Waters Acquity PDA Detector under the following conditions:

| | |
|---|---|
| Sampling rate (points/sec) | 5 |
| Wavelength range (nm) | 200-490 |
| Resolution (nm) | 3.6 |

The mass spectrometer used was Waters Xevo G2-XS QTof under the following conditions:

| Ionization mode | Electrospray Positive |
|---|---|
| Acquisition mode | MSe |
| Mass range (Da) | 80-1000 |
| Scan time (s) | 0.150 |
| Capillary voltage (kV) | 0.5 |
| Sample cone (V) | 30 |
| Low collision energy (eV) | 4 |
| High collision energy ramp range (eV) | 20-50 |
| Source temperature (° C.) | 120 |
| Desolvation temperature (° C.) | 500 |
| Cone gas flow (L/h) | 50 |
| Desolvation gas flow (L/h) | 1200 |

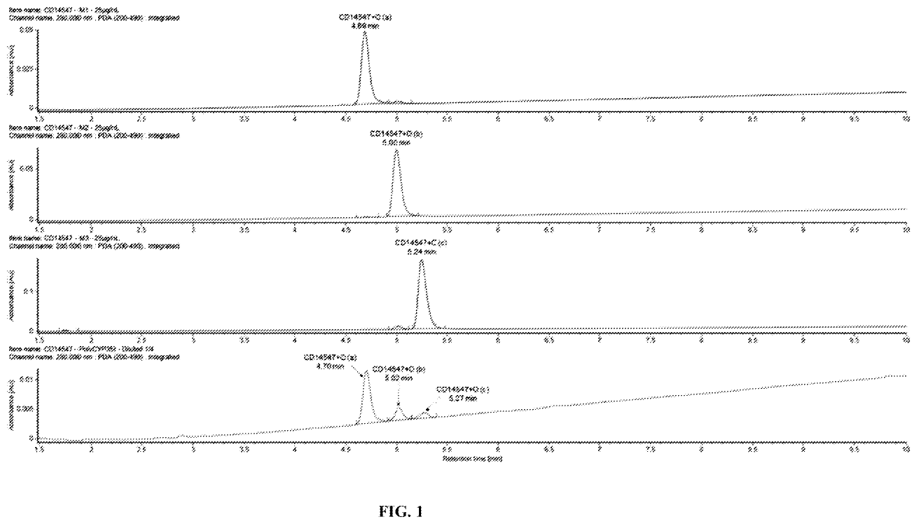

The LC-UV-HRMS analysis confirmed the content of the compounds CD14547 M1, CD14547 M2, CD14547 M3 (FIG. 1) and of the pools A, B and C compare to the PolyCYP359 sample from the study 7013606.SA.285

The percentages of each compound identified in the compounds CD14547 M1, CD14547 M2, CD14547 M3 and in the pools A, B and C with the UV detection at the wavelength of 280 nm were calculated and reported in the Table 2 below.

The formulae used is:

$$\frac{Compound peak area at 280 nm}{Sum of compounds peak area at 280 nm} \times 100$$

TABLE 2
Percentages of each compound

| | CD14547 + O | | | Pool | | |
|---|---|---|---|---|---|---|
| Compounds | M1 | M2 | M3 | A | B | C |
| CD14547 | nd | nd | nd | nd | nd | nd |
| CD14547 + O (a) * | 97.5 | 0.7 | 0.2 | 1.4 | 4.6 | nd |
| CD14547 + O (b) * | 2.5 | 99.3 | 4.0 | 0.9 | 6.7 | nd |
| CD14547 + O (c) * | nd | nd | 95.8 | nd | nd | nd |
| CD14547 + O—H₂ (c) * | nd | nd | nd | nd | 4.8 | nd |
| CD14547 + O—H₂ (d) * | nd | nd | nd | nd | 37.7 | nd |
| CD14547 + O—H₂ (h′) | nd | nd | nd | 27.4 | nd | nd |
| CD14547 + O—H₂ (i′) | nd | nd | nd | 9.1 | nd | nd |
| CD14547 + O—H₂ (j′) | nd | nd | nd | 61.2 | nd | nd |
| CD14547-H₂ (a) * | nd | nd | nd | nd | 41.0 | 84.7 |
| CD14547-H₂ (b) * | nd | nd | nd | nd | 5.2 | 15.3 |

* Compounds observed in plasma samples from the in vivo PK rat study (based on relative retention time)

### Example 5: Sample Analysis

Pools A and B were solubilized at 1 mM in DMSO. The concentration of 1 mM is based on the amount of the compound present in highest quantity in each pool and considering a purity of 100%.

All solutions were then diluted to obtain solutions at 0.1 mg/mL in acetonitrile and at 25 μg/mL in MeCN/H2O (25V/75V). For the pools A and B, a centrifugation step (10 min at 13000 rpm) was added to remove the precipitate formed during the addition of the acetonitrile to prepare the solutions at 0.1 mg/mL.

The PolyCYP359 sample (sample stored at −80° C.) was thawed and its supernatant was diluted by 4 in H₂O. All solutions at 25 μg/mL and the PolyCYP359 sample diluted were analyzed with the LC-UV-HRMS method described above in order to check that the purified compounds corresponds to the compounds CD14547 M1, CD14547 M2 and CD14547 M3 and to check the content of the off-fraction pool of compounds.

### Example 6: NMR Structural Elucidation

NMR spectra were acquired on a 700 MHz instrument. Standard methods were used to acquire ¹H, ¹³C, COSY, HSQC, and HMBC NMR spectra.

The ¹H, ¹³C, COSY, HSQC, and HMBC spectra of compound CD14547 M1 in DMSO-d6 were obtained and studied. Inspection of the 1H NMR spectrum immediately indicated that the two doublet signals for the terminal methyl groups of the alkyl side chain in the CD14547 spectrum were absent, and had been replaced by a 6-proton singlet signal at 0.93 ppm. This showed an HMBC correlation with a new quaternary carbon signal at 68.5 ppm, which also shared HMBC correlations with the alkyl chain methine signal at 4.92 ppm (position 15 as shown by a COSY correlation with the methyl doublet at 1.36 ppm) and the methylene proton signals at 2.37 and 1.76 ppm (position 16, shifted downfield compared to the corresponding signals in the CD14547 spectrum) as well as with a new sharp proton singlet signal at 4.22 ppm representing an exchangeable proton (no HSQC correlation). Inspection of all the M1 NMR spectra indicated that the signals for the benzoxazole and pyrazolopyrimidine moieties were essentially unchanged compared to those of CD14547. These data indicate that M1 was formed by hydroxylation at position 17 to yield a tertiary alcohol group.

The ¹H, ¹³C, COSY, HSQC, and HMBC spectra of the parent compound, CD14547 in DMSO-d6 were obtained and studied. The assignment of the ¹H and ¹³C NMR signals to the structure of CD14547 was straightforward. The ¹H, ¹³C, COSY, HSQC, and HMBC spectra of compound CD14547 M2 in DMSO-d6 and those for M3 were obtained and studied. It was observed that although the spectra for the two compounds are distinct, they are very similar with the only differences being for the signals representing the alkyl side chains while the signals for the benzoxazole and pyrazolopyrimidine moieties were essentially unchanged compared to those for CD14547. For both compounds it was immediately apparent that one of the terminal methyl doublet proton signals had been replaced by a pair of hydroxymethylene proton signals. For M2 these signals were doublets of doublets at 3.19 and 3.09 ppm, which showed an HSQC correlation to a carbon signal at 66.6 ppm; for M3 the new doublets of doublets were at 3.28 and 3.23 ppm showing an HSQC correlation to a carbon signal at 65.5 ppm. For both M2 and M3 these new hydroxymethylene groups were shown by COSY and HMBC correlations to be adjacent to the 17-methine group, which had signals at 1.15/32.5 ppm for M2 and 1.38/32.7 ppm for M3. These carbon shifts are downfield to that of CD14547 by the same amount. These data indicate that CD14547 compounds M2 and M3 are atropisomers formed by hydroxylation of the non-equivalent terminal methyl groups of the alkyl side chain of CD14547.

### Example 7: Chemical Synthesis of Compounds: Characterization Methods

In the synthetic process described below, the reaction products were isolated and characterized using liquid chromatography/mass spectrometry (LC/MS), and Nuclear magnetic resonance (¹H NMR and ¹³C NMR). Chiral supercritical fluid chromatography (SFC) (ChiralPak IC column) was used to separate and analyze the mixture of regioisomers in order to obtain enantiomers.

### (A) LC/MS

LC/MS were recorded using 2 different methods and apparatus as described below:

Method A was recorded on Waters UPLC Acquity system using the following parameters:
Column: Acquity BEH C18 1.7 m 2.1*150 mm;
5 min runs
Photodiode array detection from 210 to 400 nm
Solvent system:
   Mobile phase A: Ammonium carbonate aqueous solution 2 g/L
   Mobile phase B: MeCN
Gradient:

| Time (min) | Flow (ml/min) | % A | % B |
|---|---|---|---|
| 0 | 0.50 | 95 | 5 |
| 0.20 | 0.50 | 95 | 5 |
| 3.30 | 0.50 | 5 | 95 |
| 4.50 | 0.50 | 5 | 95 |
| 4.70 | 0.50 | 95 | 5 |
| 5.00 | 0.50 | 95 | 5 |

Method B was recorded on Waters UPLC Acquity system using the following parameters:
Column: Acquity CSH C18 1.7 m 2.1*150 mm;
30 min runs
Photodiode array detection from 210 to 400 nm. Detection at 284 nm.
Solvent system:
   Mobile phase A: Water+0.1% Formic Acid
   Mobile phase B: Acetonitrile+0.1% Formic Acid
Gradient:

| Time (min) | Flow (ml/min) | % A | % B |
|---|---|---|---|
| 0 | 0.35 | 95 | 5 |
| 1.0 | 0.35 | 95 | 5 |
| 25 | 0.35 | 5 | 95 |
| 28.0 | 0.35 | 5 | 95 |
| 28.1 | 0.35 | 95 | 5 |
| 30.0 | 0.35 | 95 | 5 |

### (B) Chiral Chromatography

Chiral SFC chromatography was performed with a Waters UPC2 coupled with a Diode Array and QDa detector.
Column: Chiralpak AD-3 150*4.6 mm, 3 m
Flow rate: 2 ml/mn
Sample concentration: 1 mg/mL (Methanol)
Temperature oven: 40° C.
Detection: PDA detector acquisition (220-400 nm)
Injection volume: 1 μL
ABRP pressure: 2000 psi
Gradient: Isocratic CO₂ (73%)/Ethanol (27%)
Run time: 15 min.
   (C) ¹H NMR and ¹³C NMR

¹H NMR and ¹³C NMR were recorded on a Bruker Avance 400 MHz, 100 MHz. Chemical shifts are expressed in parts per million (ppm) downfield from residual solvent peaks, and coupling constants are reported in hertz (Hz).

### Example 8: Preparation of Compound of Formula II (M1/CD17716)

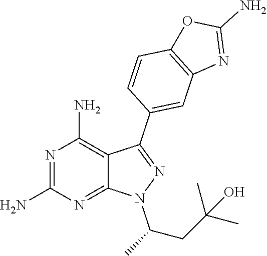 [SMILES: C[C@@H](CC(C)(C)O)n1nc(-c2ccc3oc(N)nc3c2)c2c(N)nc(N)nc21]

### Step 1: Mesylation of (R)-2-methylpentane-2,4-diol

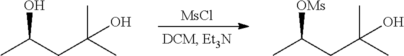

In a 10 mL flask, was charged (R)-2-methylpentane-2,4-diol (2.000 g, 0.017 mol, 1.000 eq) and DCM (14.0 mL). Under magnetical stirring and nitrogen flow was added Et₃N (3.1 mL, 0.022 mol, 1.300 eq) while cooling reaction mixture near 5° C. Then MsCl (1.4 mL, 0.019 mol, 1.100 eq) diluted in 0.5 mL of DCM was added dropwise. Quickly, a white precipitate appeared. The suspension was stirred at 23° C. for 2 h. An aliquot was taken, evaporated to dryness and analyzed by NMR (DMSO-d6). Incomplete conversion was observed and extra Et₃N (0.471 mL, 0.003 mol, 0.200 eq) was added. Stirring was pursued for 18 h. An aliquot was taken, evaporated to dryness and analyzed by NMR (DMSO-d6) and complete conversion was observed.

20 mL of water were added. The mixture was stirred for 15 minutes and phases were separated. The aqueous layer was discarded and the organic layer washed one more time with 20 mL of water, dried over magnesium sulfate, filtered and evaporated to dryness under reduced pressure to afford a pale yellow liquid.

Traces of triethylammonium chloride were still detected by NMR and the oil was dissolved in 50 mL of DCM, washed one more time with 2×20 mL of water, dried over magnesium sulfate, filtered and evaporated to dryness under reduced pressure to afford (R)-4-hydroxy-4-methylpentan-2-yl methanesulfonate (CEM-441-1; 2.710 g, 0.014 mol, 81%) as a pale yellow liquid.

### Step 2: Alkylation of 3-iodo-1H-pyrazolo(3,4-dipyrimidine-4,6-diamine

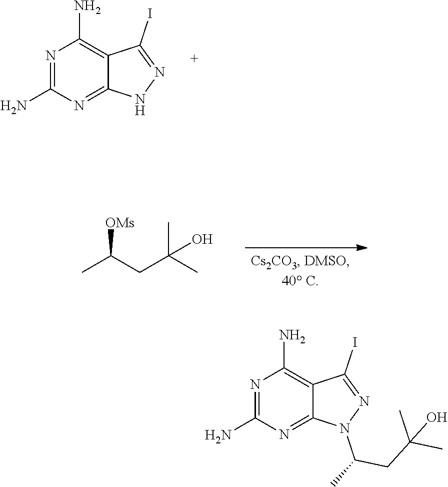 [SMILES: Nc1nc(N)c2c(I)n[nH]c2n1 ; C[C@H](CC(C)(C)O)OS(C)(=O)=O ; C[C@@H](CC(C)(C)O)n1nc(I)c2c(N)nc(N)nc21]

To a stirred solution of 3-iodo-1H-pyrazolo[3,4-d]pyrimidine-4,6-diamine (2.900 g, 0.011 mol, 1.000 eq) and CEM-441-1 (2.680 g, 0.014 mol, 1.300 eq) in DMSO (25.8 mL), was added Cs₂CO₃ (4.450 g, 0.014 mol, 1.300 eq). The suspension was heated to 40° C. and stirred for 24 h at this temperature.

An aliquot was taked after this time, diluted with MeOH and analyzed by LCMS (method A). The reaction was quenched to minimize elimination of target compound to the corresponding alkene. 2-MeTHF (50 mL) and water (50 mL) were added. Phases were separated and the aqueous layer was extracted with 50 mL of 2-MeTHF. Combined organic layers were washed with 10 mL of 0.05N HCl, dried over MgSO₄ and evaporated to dryness under vacuum to afford a pale orange oil (2.6 g). The oil was purified by flash chromatography (SiO₂, DCM/acetone 1:1) and the purest fraction were collected and combined to afford (R)-4-(4,6-diamino-3-iodo-1H-pyrazolo[3,4-d]pyrimidin-1-yl)-2-methylpentan-2-ol (CEM-443-1; 1.640 g, 0.004 mol, 39%).

### Step C: Suzuki Coupling

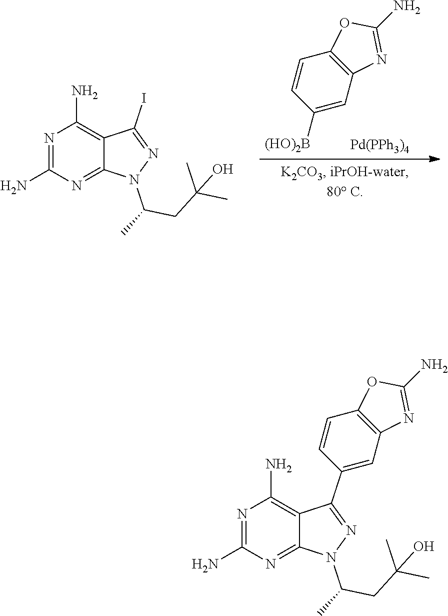 [SMILES: C[C@@H](CC(C)(C)O)n1nc(I)c2c(N)nc(N)nc21 ; Nc1nc2cc(B(O)O)ccc2o1 ; C[C@@H](CC(C)(C)O)n1nc(-c2ccc3oc(N)nc3c2)c2c(N)nc(N)nc21 ; "Pd(PPh3)4"]

In a 10 mL flask, were mixed CEM-443-1 (1.200 g, 0.003 mol, 1.000 eq) (2-aminobenzo[d]oxazol-5-yl)boronic acid (1.540 g, 0.011 mol, 3.500 eq) followed by iPrOH (12.0 mL) and water (6.0 mL). N₂ was bubbled through this solution for 15 minutes and then Pd(PPh3)4 (90 mg, 0.08 mmol, 0.025 eq) and K₂CO₃ (0.890 g, 0.004 mol, 1.300 eq) were added. The suspension was warmed to 85° C. and stirred at this temperature for 18 h. LCMS analysis (method B) of an aliquot showed complete conversion after this time. The suspension was filtered through dicalite and then 25 mL of MeTHF and 25 mL of water were added. Phases were separated and the organic layer was washed with water 20 mL. MeTHF was swapped for iPrOAc while a solid crystallized.

The solid was dissolved in 1N HCl (100 mL) and the aqueous layer was washed with TBME. Then pH of the aqueous layer was adjusted to 10 with 1N NaOH and the aqueous layer was extracted with 2-MeTHF (3×50 mL). The combined organic layers were evaporated to dryness to afford a beige solid (850 mg). This solid was purified by flash chromatography (SiO₂, DCM/MeOH 4:1) and the purest fractions were collected and combined. The solid obtained was then dissolved in acetone, filtered and evaporated to dryness under vacuum to afford Compound of formula II, (S)-4-(4,6-diamino-3-(2-aminobenzo[d]oxazol-6-yl)-1H-pyrazolo[3,4-d]pyrimidin-1-yl)-2-methylpentan-2-ol, (CEM-448-1; 560 mg, 1.46 mmol, 46%).

### Example 8: In Vitro Pharmacology Assay

A431 cells were seeded at 25000 cells/well (96 format) and the following day starved in order to synchronize all cells into the same cycle phase. The day of the experiment cells were stimulated by EGF (80 nM) and compounds (dose response) were incubated in duplicate for 3 hours before AKT phosphorylation was measured by an HTRF kit following manufacturer guidelines.

For the parent compound CD14547, the compounds CD14547 M1, CD14547 M2, CD14547 M3 and the pool C, the highest concentration tested was 1000 nM. The 1000 nM solutions were prepared from the 10 mM DMSO stock solutions described above in sample analysis section.

For the pools A and B, the highest concentration tested was 100 nM. The 1000 nM solutions were prepared from the 1 mM DMSO stock solutions described above. As each pool contains several compounds and as the amount of each compound was not precisely determined, the concentration dose response was expressed in percentage.

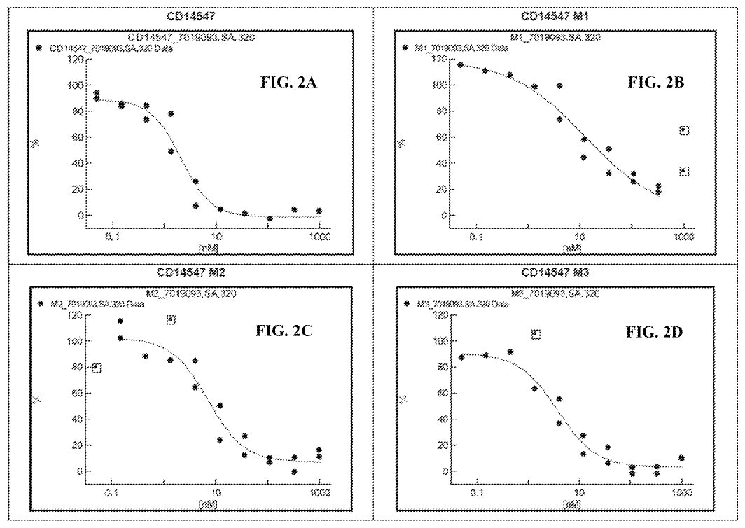

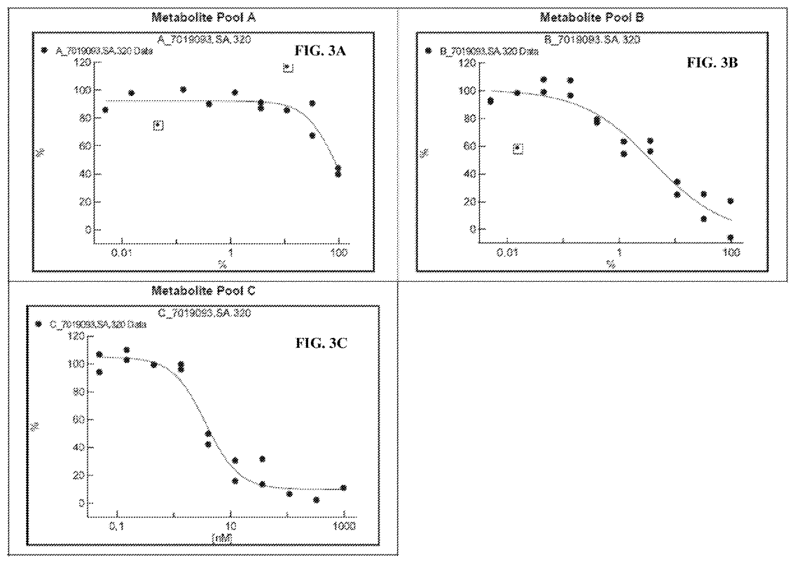

The assay was validated as no inhibition was observed with DMSO. The results of the assay (IC₅₀) are reported in the Table 3 below and the graphs are provided in FIG. 2 and FIG. 3.

TABLE 3
Results of the pharmacological activity assay

| Compound | IC₅₀ (nM) | IC₅₀ (%) | % Inhibition |
|---|---|---|---|
| CD14547 | 2.0 | n/a | 90 |
| CD14547 M1 | 13 | n/a | 103 |
| CD14547 M2 | 7.0 | n/a | 95 |
| CD14547 M3 | 3.7 | n/a | 87 |
| Pool C | 3.6* | n/a | 95 |
| Pool A | n/a | 90 | 54 |
| Pool B | n/a | 3.9 | 105 |

* Based on CD14547-H₂ (a) amount

The compounds CD14547 M1, M2, M3 and the pool C modulate AKT phosphorylation (mTOR inhibition) and shared a similar low nanomolar range activity with CD14547.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

While certain embodiments have been illustrated and described, it should be understood that changes and modifications can be made therein in accordance with ordinary skill in the art without departing from the technology in its broader aspects as defined in the following claims.

What is claimed is:

1. A compound of formula (I), a pharmaceutically acceptable salt thereof, or an enantiomer thereof, 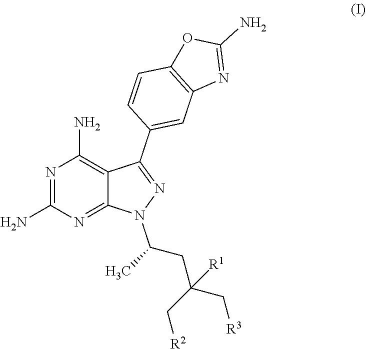
wherein:
R¹, R² and R³ are each independently selected from hydrogen or OH.

2. The compound as claimed in claim 1, having formula (II) 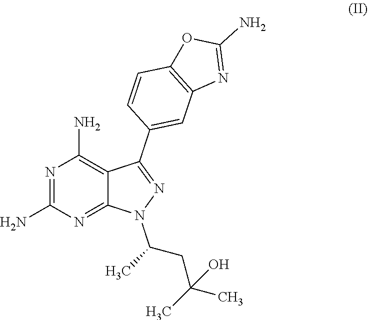 [SMILES: C[C@@H](CC(C)(C)O)n1nc(-c2ccc3oc(N)nc3c2)c2c(N)nc(N)nc21]
a pharmaceutically acceptable salt thereof, or an enantiomer thereof.

3. The compound as claimed in claim 1, having formula (III) 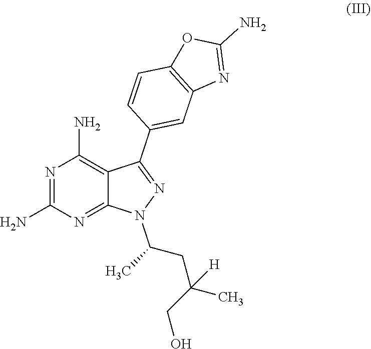 [SMILES: CC(CO)C[C@H](C)n1nc(-c2ccc3oc(N)nc3c2)c2c(N)nc(N)nc21]
a pharmaceutically acceptable salt thereof, or an enantiomer thereof.

4. The compound as claimed in claim 1, having the formula (IV): 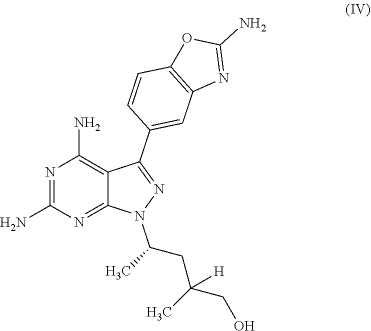 [SMILES: CC(CO)C[C@H](C)n1nc(-c2ccc3oc(N)nc3c2)c2c(N)nc(N)nc21]
a pharmaceutically acceptable salt thereof, or an enantiomer thereof.

5. The compound as claimed in claim 1, wherein when R¹ is OH, R² and R³ are each independently H.

6. The compound as claimed in claim 1, wherein when R² is OH, R¹ and R³ are each independently H.

7. The compound as claimed in claim 1, wherein when R³ is OH, R¹ and R² are each independently H.

8. A composition comprising the compound as claimed in claim 1 or a pharmaceutically acceptable salt thereof, and a pharmaceutically acceptable carrier.

9. The composition according to claim 8, comprising between 0.001% and 5% of the compound by weight relative to the total weight of the composition.

10. The composition according to claim 8, in a form suitable for oral or topical administration.

11. The composition according to claim 10, in a form suitable for topical administration.

12. A method for treating diseases involving an mTOR enzyme with serine-threonine kinase activity, comprising administering to a subject in need thereof a composition according to claim 8.

13. The method according to claim 12, for the treatment of dermatological complaints associated with a keratinization disorder with a proliferative, inflammatory and/or immunoallergic component.

14. The method according to claim 13, wherein the dermatological complaint is selected from psoriasis, atopic dermatitis, actinic keratosis or acne.

15. The method according to claim 12, for the treatment of atopic dermatitis.

16. The method according to claim 15, for the treatment of the inflammatory component of atopic dermatitis.

17. The method according to claim 15, for reinforcing the barrier function in a patient suffering from atopic dermatitis.

18. A method of treating a disease, disorder, or condition involving mTOR production, wherein the method comprises administering to a subject, the pharmaceutical composition as claimed in claim 8 to inhibit the production of mTOR in the subject.
